(19) United States
(12) Patent Application Publication
(10) Pub. No.: US 2005/0203183 A1
(43) Pub. Date: Sep. 15, 2005

(54) Compound and processes for the preparation and use thereof

(75) Inventor: Jonathan William Wiffen, Huddersfield (GB)

Correspondence Address: MORGAN LEWIS & BOCKIUS LLP, 1111 PENNSYLVANIA AVENUE NW, WASHINGTON, DC, 20004, US

(73) Assignee: AVECIA LIMITED, Manchester (GB)

(21) Appl. No.: 10/514,128

(22) PCT Filed: May 20, 2003
(86) PCT No.: PCT/GB03/02172

§ 371 Date: Nov. 12, 2004

(30) Foreign Application Priority Data
May 22, 2002 (GB) 0211751.3

(51) Int. Cl. C07F 9/70; C07F 9/90; C07F 9/94; A61K 31/225

(52) U.S. Cl. 514/548; 562/1; 556/64; 560/1

(57) ABSTRACT

A compound of Formula (1) is provided wherein X represents an optionally substituted hydrocarbyl linking group; R′ represents an optionally substituted hydrocarbyl group, P¹ and P² are protecting groups, Q represents a group of formula Y, —OZ, or —SZ; Y represents a group forming a Wittig reagent, a P, As or Sb-containing Horner-Wadsworth Emmons or Warren reagent, a P(III), As (III) or Sb(III) precursor of a Horner-Wadsworth Emmons or Warren reagent or an ylid precursor, and Z represents a group forming a P(III), As (III) or Sb(III) precursor of a Horner-Wadsworth Emmons or Warren reagent, or a P(V), As (V) or Sb(V) leaving group.

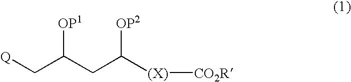

The present invention concerns certain intermediate compounds, processes for their preparation and processes for the use thereof.

According to a first aspect of the present invention, there is provided a compound of Formula (1) 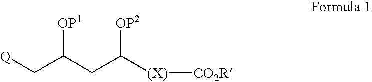
wherein
X represents an optionally substituted hydrocarbyl linking group
R′ represents an optionally substituted hydrocarbyl group,
P¹ and P² are protecting groups,
Q represents a group of formula Y, —OZ, or —SZ;
Y represents a group forming a Wittig reagent, a P, As or Sb-containing Horner-Wadsworth Emmons or Warren reagent, a P(III), As(III) or Sb(III) precursor of a Horner-Wadsworth Emmons or Warren reagent or an ylid precursor, and
Z represents a group forming a P(III), As(III) or Sb(III) precursor of a Horner-Wadsworth Emmons or Warren reagent, or a P(V), As(V) or Sb(V) leaving group.

Hydrocarbyl groups represented by X and R′ may be substituted by one or more substituents, and may be per-substituted, for example perhalogenated. Examples of substituents include halo, especially fluoro and chloro, alkoxy, such as C₁₋₆alkoxy, including C₁₋₆ branched alkoxy, and oxo.

Preferably, X represents an optionally substituted C₁₋₄alkylene group, particularly a group of formula —(CH₂)ₙ— where n is from 1 to 4, and most preferably X represents a group of formula —(CH₂)₂—.

R′ may represent an aryl group, such as a phenyl group, which may be substituted by one or more substituents. Particularly preferably, R′ represents a C₁₋₆ alkyl group, which may be linear or branched, and may be substituted by one or more substituents. Most preferably, R′ represents a t-butyl group.

Protecting groups that can be represented by P¹ or P² include such protecting groups as are commonly employed to protect hydroxy groups, for example those protecting groups disclosed in Protecting Groups in Organic Synthesis Green & Wuts; Publ. Wiley, incorporated herein by reference. Examples of protecting groups include benzyl groups, tetrahydropyranyl groups and trialkylsilyl groups, such as tri-C₁₋₄-alkylsilyl, especially t-butyldimethylsilyl groups. In many preferred embodiments, P¹ and P² together form a protecting group for 1,3-dihydroxy moieties such as a ketal, preferably an acetonide, group or a carbamate. It is most preferred that P¹ and P² together represent a group of formula>C(CH₃)₂.

When Y or Z represents a P(III), As(III) or Sb(III) precursor of a Horner-Wadsworth Emmons or Warren reagent, it will be recognised that such compounds can be converted into a Horner-Wadsworth Emmons or Warren reagent by means known in the art, for example by oxidation and/or by rearrangement.

When Y represents an ylid precursor, it will be recognised that such groups can be converted into an ylid by means known in the art, for example by reaction with a base, preferably a strong base, to remove a proton, and hence form an ylid. Such processes and the ylids so formed form another embodiment of the present invention. Bases which can be employed to produce ylids are well known in the art, and include for example an amine base such as pyridine, triethylamine or diisopropylethylamine; an alkaline hydroxide, for example ammonium hydroxide or an alkali metal hydroxide, such as is sodium hydroxide; an alkaline carbonate such as sodium or potassium carbonate; a strong base such as an alkyl lithium compound; an alkali metal amine for example sodium, potassium or lithium amine, or lithium diisopropylamide; a diazabicyclic base for example 1,8-diazabicyclo[5.4.0]undec-7-ene (DBU); an alkoxide salt such as an alkali metal alkoxide; a metal hydride such as NaH; an alkyl lithium salt such as BuLi or an alkali metal 1,1,1,3,3,3-hexamethyldisilazane salt.

Groups which can be represented by Z include groups of formula -ER₂, -E(OR)₂, -E(R)(OR), -E(SR)₂, -E(R)(SR), -E(OR)(SR), -EO(R)₂, -EO(OR)₂, -EO(R)(OR), -EO(R)(SR), -EO(OR)(SR), -EO(SR)₂, -ES(R)₂, -ES(OR)₂, -ES(R)(OR), -ES(R)(SR), -ES(SR)(OR) or -ES(SR)₂, wherein E represents P, As or Sb and each R independently represents hydrogen or an optionally substituted hydrocarbyl group, such as a C₁₋₆ alkyl or phenyl group. P(V), As(V) or Sb(V) leaving groups which can be represented by Z include groups of formula -EO(R)₂, -EO(OR)₂, -ES(R)₂, -ES(OR)₂, -EO(SR)₂ and -ES(SR)₂ wherein R is as previously defined. Such leaving groups are displaceable by nucleophiles, particularly by carbanion-type nucleophiles, such as organometallic nuclephiles, for example organolithium salts, and Grignard-type reagents. Preferred groups which can be represented by Z include —PR₂; —P(OR)₂; —P(SR)₂; —PO(R₂); —PS(R₂); —PO(OR)₂; —PS(OR)₂; and —PS(SR)₂ wherein each R independently represents hydrogen or an optionally substituted C₁₋₆alkyl, or optionally substituted aryl, especially a phenyl, group. The most preferred group which can be represented by Z is —PPh₂.

Groups which can be represented by Y include those groups above which can be represented by Z and groups of formula —(S═O)R, —(SR₂)⁺X⁻, —(NR₃)⁺X⁻, PR₃⁺X⁻, SbR₃⁺X⁻ and AsR₃⁺X⁻ in which each R independently represents hydrogen or an optionally substituted hydrocarbyl group, preferably an optionally substituted C₁₋₆alkyl or optionally substituted phenyl group; and X represents a monovalent anion, preferably a halide ion, and especially an iodide ion.

Preferably, Q represents a group of formula Y in which Y represents a group of formula —PR₂, P(OR)₂, —PO(OR)₂ or —PO(R₂) wherein each R independently is hydrogen or optionally substituted C₁₋₆ alkyl, especially methyl, ethyl, isopropyl or sec-butyl, or optionally substituted phenyl.

Preferred compounds according to the present invention are compounds of Formula 2: 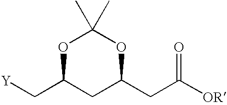
wherein
R′ represents a C₁₋₆ alkyl group, most preferably a t-butyl group; and
Y represents —P(OR)₂, —PR₂, —PO(OR)₂ or —PO(R₂) wherein each R independently is optionally substituted C₁₋₆ alkyl, especially methyl, ethyl, isopropyl or sec-butyl, or optionally substituted phenyl.

Especially preferred compounds according to the present invention are those compounds of Formula 2 in which Y represents —PO(OCH₃)₂; —PO(OC₂H₅)₂; —PO(OCH(CH₃)₂)₂ or —PO(Ph)₂.

According to the second aspect of the present invention, there is provided a process for the preparation of a compound of Formula (1) 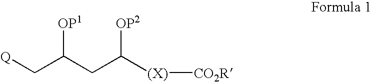
which comprises reacting a compound of Formula (3) 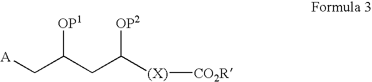
wherein
X, R′, P¹, P² and Q are as described above;
A represents OH, O(COR^{z}) or a leaving group; and
R^{z} represents an optionally substituted hydrocarbyl group, preferably a C₁₋₆ alkyl group; with, depending on the nature of Q, a reagent suitable for the formation of a Wittig reagent, a P, As or Sb-containing Horner-Wadsworth Emmons or Warren reagent, a P(III), As(III) or Sb(III) precursor of a Horner-Wadsworth Emmons or Warren reagent, a P(V), As(V) or Sb(V) leaving group or an ylid precursor.

Examples of leaving groups that can be represented by A include halogen, especially Cl, Br and I, optionally substituted aryl or alkyl sulphonates especially tosylate, brosylate, mesylate, trifluoromesylate and triflate. The reaction takes place under conditions known in the art for the formation of the given moiety represented by Q, depending for example on the selected groups A for the chosen compound of Formula 3 and the chosen reagent. Commonly, the reaction takes place in the presence of an inert organic solvent. Both polar and non-polar solvents may be employed, particularly aprotic solvents, and examples include hydrocarbons, especially toluene, chlorocarbons, especially dichloromethane and chloroform, nitriles, such as acetonitrile, ethers, including dioxane and tetrahydrofuran, amides such as dimethylformamide. Preferably, substantially anhydrous conditions are employed.

For the preparation of compounds of Formula 1 wherein Q represents OZ or SZ, and Z represents a group comprising a trivalent group E, for example of formula ER₂, E(OR)₂, or E(SR)₂ as hereinbefore defined, a compound of Formula 3 wherein A represents —OH or —SH can be reacted with a compound of formula D-Z, wherein D is a displaceable group, commonly a group R, OR, SR or a halogen, especially chlorine or bromine. The reaction preferably takes place under basic conditions, for example in the presence of an amine base such as pyridine, triethylamine or diisopropylethylamine; an alkaline hydroxide, for example ammonium hydroxide or an alkali metal hydroxide, such as sodium hydroxide; an alkaline carbonate such as sodium or potassium carbonate; a strong base such as an alkyl lithium compound; an alkali metal amine for example sodium, potassium or lithium amine, or lithium diisopropylamide; a diazabicyclic base for example 1,8-diazabicyclo[5.4.0]undec-7-ene (DBU); a metal hydride such as NaH; an alkoxide salt such as an alkali metal alkoxide; an alkyl lithium salt such as BuLi or an alkali metal 1,1,1,3,3,3-hexamethyldisilazane salt.

Compounds of Formula 1 in which Q represents Y, and Y represents a group comprising a pentavalent moiety E, for example of formula EO(R)₂, EO(OR)₂, ES(R)₂, ES(OR)₂, EO(SR)₂ or ES(SR)₂ as hereinbefore defined, can be prepared by rearrangement of the corresponding compound wherein Q represents OZ or SZ, and Z represents a the corresponding group comprising the trivalent group E, for example, of formula ER₂, E(OR)₂, or E(SR)₂. Such a rearrangement may be effected by treatment with a nucleophile, heating or reaction with a free radical. Examples of such conditions are given in Chem Rev (1984) 84 577 and J Am Chem Soc (1959) 81 1243, such conditions being incorporated herein by reference.

Compounds of Formula 1 in which Q represents Y, and in which Y represents —(SR₂)⁺X⁻ and —(NR₃)⁺X⁻, (PR₃)⁺X⁻ and the corresponding As and Sb compounds can be prepared by reaction between a compound of Formula 3 in which A represents halogen, especially iodine, and a compound of formula R₂S, a quaternary ammonium salt or a compound of formula PR₃ (or the corresponding As or Sb compounds), where R is preferably C₁₋₆ alkyl or aryl, and most preferably PPh₃. When a reagent of formula R₂S or a compound of formula PR₃ (or the corresponding As or Sb compounds) is employed, the reaction takes place in the presence of a base, for example an amine base such as pyridine, triethylamine or diisopropylethylamine; an alkaline hydroxide, for example ammonium hydroxide or an alkali metal hydroxide, such as sodium hydroxide; an alkaline carbonate such as sodium or potassium carbonate; a strong base such as an alkyl lithium compound; an alkali metal amine for example sodium, potassium or lithium amine, or lithium diisopropylamide; a diazabicyclic base for example 1,8-diazabicyclo[5.4.0]undec-7-ene (DBU); a metal hydride such as NaH; an alkoxide salt such as an alkali metal alkoxide; an alkyl lithium salt such as BuLi or an alkali metal 1,1,1,3,3,3-hexamethyldisilazane salt. When a quaternary ammonium compound is employed, the reaction takes place in the presence of a strong base, such as those described above.

In a preferred embodiment of the second aspect of the present invention, a compound of Formula 2 as defined above in which Y represents —PO(R)₂ is prepared by reaction between a compound of formula 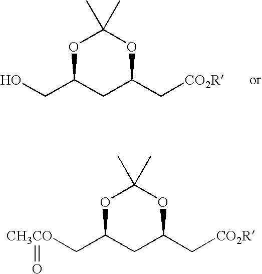
wherein:
R′ is as described previously;
and a compound of formula PR₃ or P(OR)₃ wherein each R, independently, is hydrogen or an optionally substituted alkyl, preferably C₁₋₆ alkyl, or optionally substituted phenyl group.

In another preferred embodiment of the second aspect of the present invention, a compound of Formula 2 wherein A represents —OH is reacted with a compound of formula R₂P-T, wherein T represents a halogen, preferably Cl, and R represents an optionally substituted alkyl or optionally substituted phenyl group. A preferred compound of formula R₂P-T is Ph₂PCl.

Compounds of Formula 1 are useful synthons for the production of valuable pharmaceutical compounds. In particular, they are useful for the synthesis of a wide range of compounds having HMG-CoA inhibitory action, commonly known as statins. Such compounds commonly comprise a 3,5-dihydroxyhexanoic acid moiety linked via a carbon-carbon double bond to a bulky, typically cyclic, organic group. The compounds of Formula 1 can be employed to couple a 3,5-dihydroxyhexanoic acid moiety, or a protected precursor thereof, to an organic moiety by the formation of a carbon-carbon double bond by reaction with an aldehyde group.

Accordingly, according to a third aspect of the present invention, there is provided a process for the preparation of a compound of formula 4: 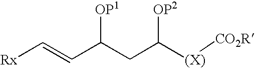
wherein R′, X, P¹ and P² are as described previously;
which comprises reacting a compound of formula RxCH═O, wherein Rx represents H or an organic radical, with either a compound of Formula 1 in which Q represents Y, and Y represents a group forming a Wittig, Horner Wadsworth Emmons or Warren reagent, or an ylid obtained from a compound of Formula 1 when Q represents Y and Y is an ylid precursor.

In many embodiments, Rx comprises one, two or more rings, preferably 5 or 6 membered-rings, often comprising at least one cyclic or heterocyclic aromatic group, commonly comprising a 5 or 6 membered aromatic ring, which may be substituted by one or more substituents. Such substituents include one or more cyclic groups, which may form a conjugated bicyclic ring system, one or more aryl substituents, especially phenyl substituents, which may themselves be substituted, and one or more alkyl substituents, including cycloalkyl substituents. Commonly, a Rx is a heteroaromatic group, often comprising one or two heteroatoms, most commonly nitrogen atoms.

The process of the third aspect of the present invention commonly takes place in the presence of a base such as an alkyl lithium, a metal, such as an alkali metal, hydride, an alkoxide salt such as an alkali metal alkoxide, alkali metal amine base for example sodamide or lithium diisoproplyamide, a diazabicyclic base for example 1,8-diazabicyclo[5.4.0]undec-7-ene (DBU) an alkali metal 1,1,1,3,3,3-hexamethyldisilazane salt or an alkali metal hydroxide.

An inert organic solvent is commonly employed. Both polar and non-polar solvents may be employed, and examples include hydrocarbons, especially toluene, chlorocarbons, especially dichloromethane and chloroform, nitriles, such as acetontirile, ethers, including dioxane and tetrahydrofuran, amides such as dimethylformamamide, and ketones, such as acetone. Preferably, substantially anhydrous conditions are employed.

Examples of compounds of formula RxCH═O which can be employed in the process according to the third aspect of the present invention are compounds in which Rx represents one of:

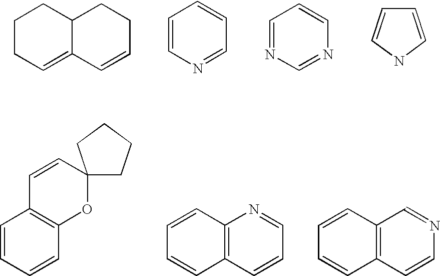

The heterocycles given for Rx may be substituted at any available position by the —CHO group, and further substituted by one or more substituents. The bonding within the ring system may be as shown or single/double at any position within the ring.

According to a fourth aspect of the present invention, there is provided a process for the preparation of a compound of Formula 5 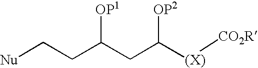
wherein P¹, P², X and R′ are as previously described, which comprises reacting a compound of Formula 1 wherein Q represents —OZ or —SZ, and Z represents a P (V), As(V) or Sb (V) leaving group, with a nucleophile comprising a moiety Nu. Preferred nucleophiles are compound of formula [Rx-(CRyRz)]ₙM, wherein M represents a metallic group of valency n, where n is 1 or 2, such as lithium, sodium, potassium, calcium, tin, a Grignard metal salt, for example MgCl, MgBr, or Mgl, or the copper or zinc compound produced by transmetallation of a Grignard metal salt, Rx is as described above, and Ry and Rz are each independently H, hydrocarbyl groups, such as to C₁₋₄ alkyl groups, or leaving groups, provided that both of Ry and Rz are not leaving groups. Preferred leaving groups are groups capable of elimination to form a carbon-carbon double bond. Preferably either both Ry and Rz are H, or Ry is H and Rz represents halo, or a protected hydroxy group Protecting groups for hydroxy are well known in the art, and include mesyl, tosyl, silyl, sulphonyloxy, triflate, nonaflate, tresylate, benzoyl, benzyl, THP and acetoxy groups. The protecting groups may be removed to form a free hydroxy group after the nucleophilic reaction. When either of Ry or Rz is a leaving group capable of elimination to form a double bond, the compounds of Formula 5 may be converted to compounds of Formula 4.

In the aspects of the present invention, substituents which may be present include linear or branched alkyl, such as C₁₋₆ alkyl, groups, aryl groups, especially phenyl groups, cycloalkyl, commonly C₃₋₇ cycloalkyl groups, halo groups, especially fluoro, chloro and bromo groups, alkoxy, such as C₁₋₆ alkoxy groups, aryloxy, especially phenoxy groups, amino, amido and imino groups, oxo groups, nitro groups, sulpho groups, sulphonamido groups. Such substituents may themselves bear one or more substituents, for example alkyl and particularly phenyl groups are commonly further substituted with one or more substituents, especially F atoms. Aryl groups may comprise fused ring structures with further aryl, including heteroaryl, or alicyclic, rings.

The invention is further illustrated without limitation by the following examples.

### EXAMPLE 1

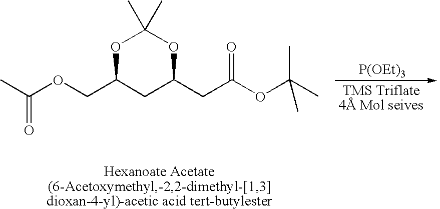

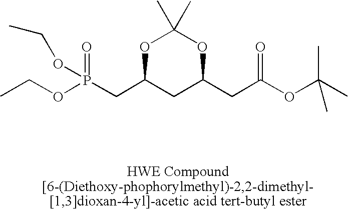

| | | Actual Wt | Strength | 100% Wt | | No | |
|---|---|---|---|---|---|---|---|
| Material | Source | (g) | (%) | (g) | MW | mmoles | Mol/mol |
| Hexanoate | | 1.0 | Ass 100 | 1.0 | 302.4 | 3.306 | 1.00 |
| Acetate | | | | | | | |
| TMS Triflate | Fisher | 0.75 | 99 | 0.73 | 222.26 | 3.284 | 1.00 |
| Triethyl | Fisher | 1.2 | 100 | 1.2 | 166.16 | 7.22 | 2.18 |
| phosphite | | | | | | | |
| 4Å mol sieves | Fisher | 1.7 | — | 1.7 | — | — | — |
| (powdered) | | | | | | | |
| Acetonitrile | Fisher | 10 ml | — | — | — | — | — |

Procedure

Hexanoate Acetate (1.0 g), Triethylphosphite (1.2 g), 4 Å molecular sieves (1.7 g) and acetonitrile were charged to a 25 ml flask and stirred at ambient for 10 minutes. The slurry was cooled to 0° C. and TMS Triflate (0.75 g) was added dropwise. The mass was is stirred for 1 hour and a sample was taken for ¹H and ³¹P NMR analysis which confirmed the formation of the Hexanoate HWE compound given above.

### EXAMPLE 2

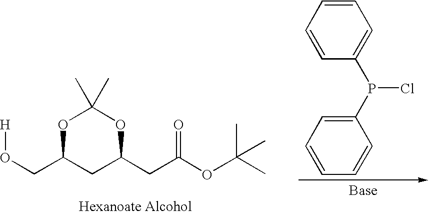

| | 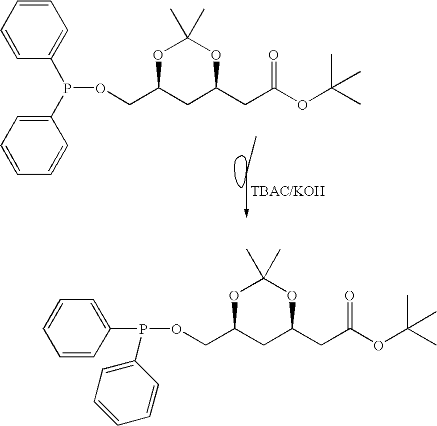 | | | | | | |
|---|---|---|---|---|---|---|---|
| | | Actual Wt | Strength | 100% Wt | | No | |
| Material | Source | (g) | (%) | (g) | MW | mmoles | Mol/mol |
| Hexanoate | | 2.24 | 100 | 2.24 | 260.3 | 8.61 | 1.0 |
| Alcohol | | | | | | | |
| Triethylamine | Fisher | 0.88 | 99 | 0.88 | 101.2 | 8.7 | 1.0 |
| Chlorodiphenyl | Acros | 2.0 | 95 | 1.9 | 220.64 | 8.61 | 1.0 |
| phosphine | | | | | | | |
| Methylene | Fisher | 20 ml | — | — | — | — | — |
| Chloride | | | | | | | |
| Aq KOH | Fisher | 3.56 | 20 | 0.71 | 56 | 12.8 | 1.5 |
| Tetrabutyl | Acros | 0.24 | 100 | 0.24 | 277.92 | 0.861 | 0.1 |
| ammonium | | | | | | | |
| chloride | | | | | | | |
| Toluene | Fisher | 16 ml | — | — | — | — | — |

Procedure Step 1

Hexanoate alcohol (2.24 g), DCM (20 ml) and triethylamine (0.88 g) were charged to a 50 ml flask and refluxed (˜40° C.) for 18 hours. The mass was subsequently cooled and the solvent removed in vacuo to afford an oil.

NMR ³¹P gave a peak at 116.6 Hz consistent with the formation of the P(III) compound given above.

Procedure Step 2

The oil obtained from Step 1 was dissolved in toluene (16 ml) and the solution heated to 60° C. Tetrabutyl ammonium chloride and potassium hydroxide solution were charged and the mixture was heated at 60° C. for a further 4 hours. The product was extracted into toluene and washed with hot water to afford the crude product.

NMR ³¹P gave a peak at 20.1 Hz consistent with the formation of the P(V) compound given above.

### EXAMPLE 3

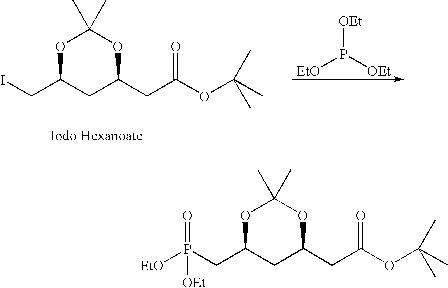

Reagents

| | Mol. Wt. | No. of moles | Mass or Volume |
|---|---|---|---|
| Iodo Hexanoate | 370 | 1.35 × 10⁻³ | 0.5 g |
| Triethylphosphite | 166.16 | | 10 ml |

Method

Iodo hexanoate was dissolved in triethylphosphite and heated under N₂ for 16 h at 150° C. Initially the solution turned a red/orange colour which then diminished to give a pale yellow solution. After 16 h the triethylphosphite is removed under reduced pressure to give a pale yellow highly viscous oil. Analysis by NMR showed complete conversion of iodo hexanoate to the Horner Wadsworth Emmonds reagent.

1. A compound of Formula (1) 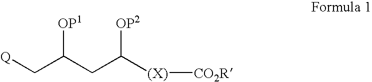
wherein
X represents an optionally substituted hydrocarbyl linking group
R′ represents an optionally substituted hydrocarbyl group,
P¹ and P² are protecting groups,
Q represents a group of formula Y, —OZ, or —SZ;
Y represents a group forming a Wittig reagent, a P, As or Sb-containing Horner-Wadsworth Emmons or warren reagent, a P(III), As(III) or Sb(III) precursor of a Horner-Wadsworth Emmons or warren reagent or an ylid precursor, and
Z represents a group forming a P(III), As (III) or Sb(III) precursor of a Horner-Wadsworth Emmons or Warren reagent, or a P(V), As(V) or Sb(V) leaving group.

2. A compound according to claim 1, wherein Q represents a group of formula Y, and Y represents a P-containing Horner-Wadsworth Emmons or Warren reagent or a P(III) precursor of a Horner-Wadsworth Emmons or Warren reagent.

3. A compound according to claim 2, wherein Y represents —P(OR)₂; —PO(OR)₂ or —PO(R)₂, wherein each R independently is an optionally substituted C₁₋₆ alkyl or an optionally substituted phenyl group.

4. P A compound according to claim 1, wherein Q represents a group of formula —OZ, and Z represents a P(III) precursor of a Horner-Wadsworth Emmons or Warren reagent.

5. A compound according to claim 4, wherein —OZ represents —OP(OR)₂ or —OP(R)₂, wherein each R independently is an optionally substituted C₁₋₆ alkyl or an optionally substituted phenyl group.

6. A compound according to any claim 1, wherein X represents —CH₂—.

7. A compound according to claim 1, of Formula 2: 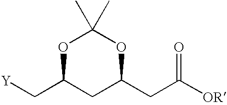
wherein
R′ represents a C₁₋₆ alkyl group, most preferably a t-butyl group; and
Y represents —P(OR)₂, —PR₂, —PO(OR)₂ or —PO(R₂) wherein each R independently is optionally substituted C₁₋₆ alkyl, especially methyl, ethyl, isopropyl or sec-butyl, or optionally substituted phenyl.

8. A process for the preparation of a compound of Formula (1) 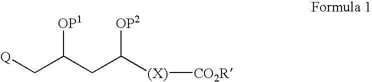
which comprises reacting a compound of Formula (3) 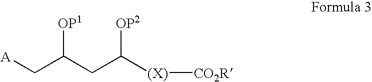
wherein
X, R′, P¹, P² and Q are as defined in claim 1;
A represents OH, O(COR^{z}) or a leaving group; and
R^{z} represents a hydrocarbyl group, preferably a C₁₋₆ alkyl group;
with, depending on the nature of Q, a reagent suitable for the formation of a Wittig reagent, a P, As or Sb-containing Horner-Wadsworth Emmons or Warren reagent, a P(III), As (III) or Sb(III) precursor of a Horner-Wadsworth Emmons or Warren reagent, a P(V), As(V) or Sb(V) leaving group or an ylid precursor.

9. A process according to claim 8, wherein a compound of formula 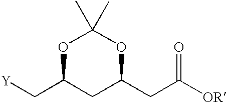
in which Y represents —PO(R)₂ and R′ represents a C₁₋₆ alkyl group, most preferably a t-butyl group, is prepared by reaction between a compound of formulae 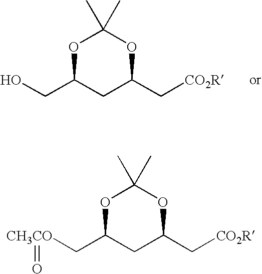
and a compound of formula PR₃ or P(OR)₃ wherein each R, independently, is H or an optionally substituted alkyl or optionally substituted phenyl group.

10. A process according to claim 8, wherein a compound of formula 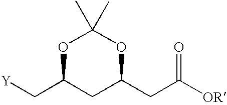
in which Y represents —PO(R)₂ and R′ represents a C₁₋₆ alkyl group, most preferably a t-butyl group, is prepared by:
a) reaction between a compound of formula 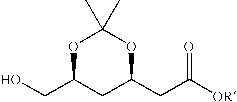
and a compound of formula R₂P-T, wherein T represents a halogen, preferably Cl, and R represents an optionally substituted alkyl or optionally substituted phenyl group; and
b) re-arranging the compound produced in step a).

11. A process according to claim 10, wherein the compound of formula R₂P-T is Ph₂PCl.

12. A process for the preparation of a compound of formula 4: 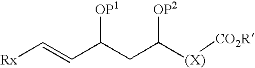
wherein R′, X, P¹ and P² are as defined in claim 1;
which comprises reacting a compound of formula RxCH═O wherein Rx represents H or an organic radical with either a compound of Formula 1 in which Q represents Y, and Y represents a group forming a Wittig, Horner Wadsworth Emmons or Warren reagent, or an ylid obtained from a compound of Formula 1 when Q represents Y and Y is an ylid precursor.

13. A process according to claim 12, wherein Rx comprises one, two or more 5 or 6 membered aromatic or heteroaromatic rings.

14. A process according to claim 12 or 13, which takes place in the presence of a base.

15. A process for the preparation of an ylid, which comprises reacting a compound of formula 1 as claimed in claim 1, and wherein Q represents Y, and Y is an ylid precursor, with a base thereby to form an ylid.

16. An ylid obtainable by a process according to claim 15.

17. A process for the preparation of a compound of Formula 5 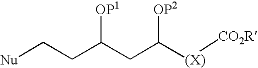
wherein P¹, P², X and R′ are as defined in claim 1, which comprises reacting a compound of Formula 1 as defined in claim 1, wherein Q represents —OZ or —SZ, and Z represents a P(V), As(V) or Sb(V) leaving group, with a nucleophile comprising a moiety Nu.

18. A process according to claim 17, wherein the nucleophile has the formula [Rx-(CRyRz)]ₙM, wherein M represents a metallic group of valency n, where n is 1 or 2, Rx is as described above, and Ry and Rz are each independently H, hydrocarbyl groups or leaving groups, provided that both of Ry and Rz are not leaving groups.

19. A process according to claim 18, wherein M is lithium, sodium, potassium, calcium, tin, a Grignard metal salt, or the copper or zinc compound produced by transmetallation of a Grignard metal salt.

20. A process according to claim 18, wherein one of Ry and Rz is a leaving group, and wherein the compound of Formula 5 is subjected to elimination thereby to form compound of Formula 4 as described in claim 12.

21. (canceled)

22. The process which comprises converting a compound according to claim 1 or an ylid according to claim 16 into a pharmaceutical.

23. The process of claim 22 wherein the pharmaceutical is a statin.
